(19) United States
(12) Patent Application Publication
(10) Pub. No.: US 2017/0197914 A1
(43) Pub. Date: Jul. 13, 2017

(54) METHOD FOR PREPARING AN AROMATIC SULFIDE OR SALT THEREOF

(71) Applicant: INDUSTRIAL TECHNOLOGY RESEARCH INSTITUTE, Hsinchu (TW)

(72) Inventors: Po-Hsien HO, Taipei City (TW); Chih-Hsiang LIN, Taipei City (TW); Meng-Hsin CHEN, Xinpi Township (TW); Cheng-Hsing FAN, Tainan City (TW); Hsin-Ching KAO, Baoshan Township (TW); Yih-Her CHANG, Baoshan Township (TW)

(73) Assignee: INDUSTRIAL TECHNOLOGY RESEARCH INSTITUTE, Hsinchu (TW)

(21) Appl. No.: 15/389,785

(22) Filed: Dec. 23, 2016

Related U.S. Application Data
(60) Provisional application No. 62/277,091, filed on Jan. 11, 2016.

(30) Foreign Application Priority Data
Dec. 20, 2016 (TW) 105142207

(51) Int. Cl. C07C 323/20; C07C 319/14; C07C 321/28

(52) U.S. Cl. CPC C07C 323/20; C07C 321/28; C07C 319/14

(57) ABSTRACT

A method for preparing an aromatic sulfide or a salt thereof is provided. The method for preparing an aromatic sulfide or a salt thereof includes reacting a compound having a structure represented by Formula (I) to a compound having a structure represented by Formula (III) in the presence of a compound having a structure represented by Formula (II) to obtain a compound having a structure represented by Formula (IV)

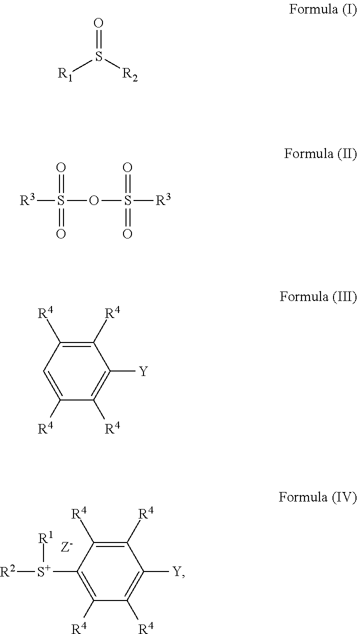

wherein R¹ and R² are independently C₁₋₆ alkyl group, or C₅₋₇ cycloalkyl group; R³ is independently C₁₋₆ alkyl group, C₅₋₇ cycloalkyl group, C₁₋₆ haloalkyl group, or aryl group; R⁴ is independently H, or C₁₋₆ alkyl group; Y is H, aryl group, or —X—R⁵; X is —O—, —NH—, —PH—, or —S—, or Y and an adjacent R⁴ are optionally combined with the carbon atoms to which they are attached, to form an aryl group; and R⁵ is H, C₁₋₆ alkyl group, C₅₋₇ cycloalkyl group, or aryl group; and Z⁻ is R³SO₃⁻.

### CROSS REFERENCE TO RELATED APPLICATIONS

This application claims the benefit of U.S. Provisional Application No. 62/277,091, filed on Jan. 11, 2016, which is incorporated herein by reference.

The application is based on, and claims priority from, Taiwan Application Serial Number 105142207, filed on Dec. 20, 2016, the disclosure of which is hereby incorporated by reference herein in its entirety.

### TECHNICAL FIELD

The disclosure relates to a method for preparing an aromatic sulfide or a salt thereof.

### BACKGROUND

Aromatic sulfides are important reactants in the field of organic synthesis. For example, an aromatic sulfide can serve as important intermediates reactants for preparing pesticides and pharmaceuticals. The main method for preparing aromatic alkyl sulfide (such as thioanisole) includes reacting thiophenol with alkyl chloride (such as methyl chloride) in alkaline conditions. Due to the high cost of thiophenol, the preparation cost of aromatic alkyl sulfide is increased.

Therefore, a novel method for preparing aromatic sulfide is needed.

### SUMMARY

According to embodiments of the disclosure, the disclosure provides a method for preparing aromatic sulfide or a salt thereof. The method includes reacting a compound having a structure represented by Formula (I) to a compound having a structure represented by Formula (III) in the presence of a compound having a structure represented by Formula (II) to obtain a compound having a structure represented by Formula (IV)

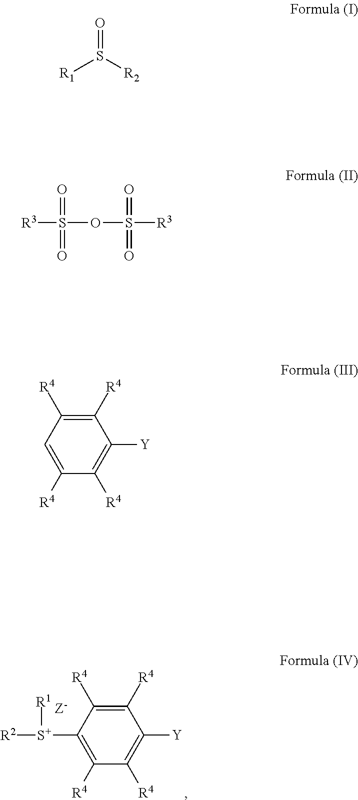

wherein R¹ and R² are independently C₁₋₆ alkyl group, or C₅₋₇ cycloalkyl group; R³ is independently C₁₋₆ alkyl group, C₅₋₇ cycloalkyl group, C₁₋₆ haloalkyl group, or aryl group; R⁴ is independently hydrogen, or C₁₋₆ alkyl group; Y is hydrogen, aryl group, or —X—R⁵, wherein X is —O—, —NH—, —PH—, or —S—, or Y and an adjacent R⁴ are optionally combined with the carbon atoms to which they are attached, to form an aryl group; and, R⁵ is hydrogen, C₁₋₆ alkyl group, C₅₋₇ cycloalkyl group, or aryl group; and Z⁻ is R³SO₃⁻.

A detailed description is given in the following embodiments with reference to the accompanying drawings.

### DETAILED DESCRIPTION

Embodiments of the disclosure provide a method for preparing aromatic sulfide or a salt thereof. Due to the use of sulfoxide as a vulcanizing agent and the use of sulfonic anhydride for activating the sulfoxide, the method for preparing aromatic sulfide or a salt thereof of the disclosure can be performed without thiophenol serving as a reactant. As a result, the preparation cost of the aromatic sulfide prepared by the method for preparing aromatic sulfide or a salt thereof of the disclosure is reduced and the yield of the aromatic sulfide prepared by the method for preparing aromatic sulfide or a salt thereof of the disclosure is improved, and there is no halogen-containing byproduct remaining in the aromatic sulfide prepared by the method for preparing aromatic sulfide or a salt thereof of the disclosure.

According to embodiments of the disclosure, the method includes reacting a compound having a structure represented by Formula (I) to a compound having a structure represented by Formula (III) in the presence of a compound having a structure represented by Formula (II) to obtain a compound having a structure represented by Formula (IV)

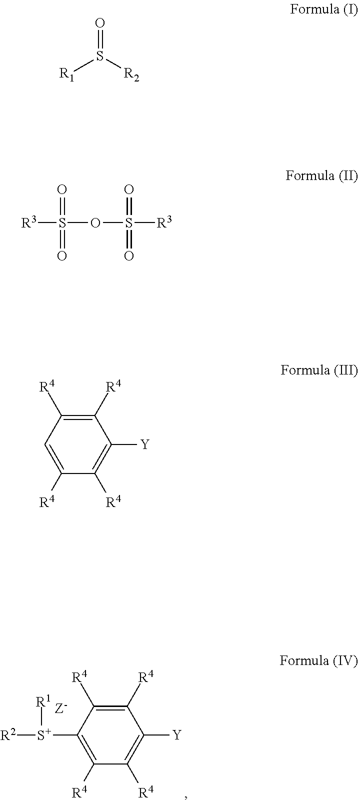

wherein R¹ and R² can be independently C₁₋₆ alkyl group, or C₅₋₇ cycloalkyl group; R³ can be C₁₋₆ alkyl group, C₅₋₇ cycloalkyl group, C₁₋₆ haloalkyl group, or aryl group; R⁴ is independently hydrogen, or C₁₋₆ alkyl group; Y is hydrogen, aryl group, or —X—R⁵, wherein X is —O—, —NH—, —PH—, or —S—, or Y and an adjacent R⁴ are optionally combined with the carbon atoms to which they are attached, to form an aryl group; and, R⁵ is hydrogen, C₁₋₆ alkyl group, C₅₋₇ cycloalkyl group, or aryl group; and Z⁻ is R³SO₃^{—}. According to embodiments of the disclosure, the compound having a structure represented by Formula (I) can serve as a vulcanizing agent. Due to the presence of the sulfonic anhydride, the sulfoxide (S═O) functional group can be protonated to form a sulfonium hydroxide functional group with reactivity, and then the sulfonium hydroxide functional group can react with the compound having a structure represented by Formula (III) to undergo an electrophilic substitution reaction to obtain an aromatic sulfide salt. In addition, a nucleophile can be reacted with the aromatic sulfide salt to undergo a dealkylation, obtaining aromatic sulfide.

According to embodiments of the disclosure, the alkyl group can be a linear or branched alkyl group. Therefore, R¹ and R² can be independently methyl, ethyl, propyl, isopropyl, n-butyl, t-butyl, sec-butyl, isobutyl, pentyl, hexyl, cyclopentyl, cyclohexyl, or cycloheptyl. In addition, According to embodiments of the disclosure, the compound having a structure represented by Formula (I) can be

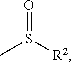

and thus the method of the disclosure can prepare aryl methyl sulfide or a salt thereof, wherein R² has the same definition as above. Moreover, according to embodiments of the disclosure, the compound having a structure represented by Formula (I) can be dimethyl sulfoxide, diethyl sulfoxide, methyl ethyl sulfoxide, methyl phenyl sulfoxide, or diphenyl sulfoxide.

According to embodiments of the disclosure, the haloalkyl group means that hydrogen atoms bonded to carbon atoms of the alkyl group can be partially or totally replaced with halogen. The haloalkyl group can be a linear or branched haloalkyl group. For example, fluoromethyl can be —CH₂F, —CHF_{2—} or —CF₃. According to embodiments of the disclosure, R³ can be independently methyl, ethyl, propyl, isopropyl, n-butyl, t-butyl, sec-butyl, isobutyl, pentyl, hexyl, fluoromethyl, fluoroethyl, fluoropropyl, cyclopentyl, cyclohexyl, or cycloheptyl. According to some embodiments of the disclosure, the compound having a structure represented by Formula (II) can be methanesulfonic anhydride, ethanesulfonic anhydride, propanesulfonic anhydride, or trifluoromethanesulfonic anhydride, or a combination thereof. According to embodiments of the disclosure, the molar ratio of the compound having a structure represented by Formula (II) to the compound having a structure represented by Formula (I) can be from about 1 to 2, such as from about 1.1 to 1.5. Herein, due to the sulfonic anhydride, an acidic condition is provided, thereby promoting the reaction of the compound having a structure represented by Formula (I) with the compound having a structure represented by Formula (III). In addition, according to embodiments of the disclosure, the excessive sulfonic anhydride can also serve as a reaction solvent.

According to embodiments of the disclosure, the molar ratio of the compound having a structure represented by Formula (III) to the compound having a structure represented by Formula (I) can be from about 1.2 to 10, such as from about 1.2 to 5. R⁴ can be independently hydrogen, methyl, ethyl, propyl, isopropyl, n-butyl, t-butyl, sec-butyl isobutyl, pentyl, or hexyl; Y can be hydrogen, phenyl, —O—R⁵, —NH—R⁵, —S—R⁵, or —PH—R⁵; and R⁵ can be hydrogen, methyl, ethyl, propyl, isopropyl, n-butyl, t-butyl, sec-butyl, isobutyl, pentyl, hexyl, cyclopentyl, cyclohexyl, cycloheptyl, or phenyl. According to embodiments of the disclosure, the compound having a structure represented by Formula (III) is

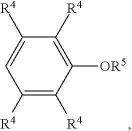

wherein R⁴ and R⁵ have the same definition as above. For example, the compound having a structure represented by Formula (III) can be benzene, biphenyl, diphenyl amine, diphenyl sulfide, or diphenyl ether.

According to embodiments of the disclosure, Y of the compound having a structure represented by Formula (III) and an adjacent R⁴ can be optionally combined with the carbon atoms to which they are attached, to form an aryl group. For example, the compound having a structure represented by Formula (III) can be naphthalene.

According to embodiments of the disclosure, the compound having a structure represented by Formula (IV) can be

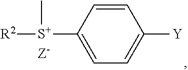

wherein R², Y, and Z⁻ have the same definition as above.

According to embodiments of the disclosure, after obtaining the compound having a structure represented by Formula (IV) (i.e. an aromatic sulfide salt), the method for preparing aromatic sulfide or a salt thereof can further include reacting a nucleophile with the compound having a structure represented by Formula (IV), obtaining a compound having a structure represented by Formula (V) (aromatic sulfide).

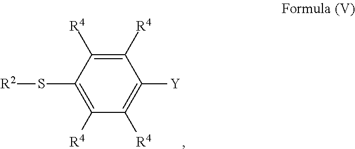

wherein R² is C₁₋₆ alkyl group, or C₅₋₇ cycloalkyl group; R⁴ is independently hydrogen, or C₁₋₆ alkyl group; Y is hydrogen, aryl group, or —X—R⁵, wherein X is —O—, —NH—, —PH—, or —S—, or Y and an adjacent R⁴ are optionally combined with the carbon atoms to which they are attached, to form an aryl group; and R⁵ is H, C₁₋₆ alkyl group, C₅₋₇ cycloalkyl group, or aryl group.

According to embodiments of the disclosure, the compound having a structure represented by Formula (V) can be

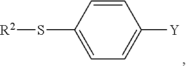

wherein R², and Y have the same definition as above. In addition, the compound having a structure represented by Formula (V) can be

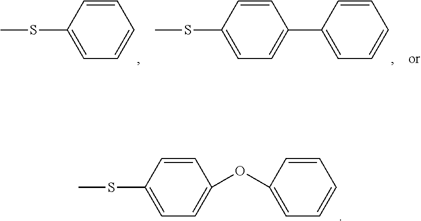

According to embodiments of the disclosure, the nucleophile can be substituted or unsubstituted pyridine or derivatives thereof (such as pyridine or 4-methylpyridine), amine (such as triethylamine), halogenated salt (such as potassium chloride), alcohol (such as methanol or ethanol), or amide (such as dimethylformamide, dimethylacetamide, or N-methylpyrrolidone). The molar ratio of the nucleophile to the compound having a structure represented by Formula (IV) can be from about 1 to 10.

The inventive concept of the disclosure may be embodied in various forms without being limited to the exemplary embodiments set forth herein.

### EXAMPLE 1

13 ml of benzene (141.5 mmol), 2 ml of dimethyl sulfoxide (28.3 mmol), and 50 ml of dichloromethane were added into a reaction bottle, and then cooled to −35° C. under a nitrogen atmosphere. After stirring for several minutes, 6 ml of trifluoromethanesulfonic anhydride (35 mmol) was added into the reaction bottle. After stirring at 0° C. (ice bath) for 1 hr, a solution with white suspension was obtained. Next, after stirring at room temperature for 12 hr, the result was concentrated, obtaining Compound 1 with a yield of about 90%. The synthesis pathway of the above reaction was as follows:

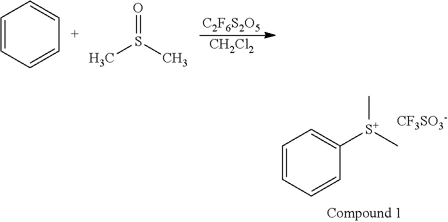

Compound 1 was analyzed by nuclear magnetic resonance (NMR) spectroscopy and the result is as follows: ¹H NMR (400 MHz, ppm, Acetone): 3.49 (—CH₃, 6H, s), 8.16-8.18 (phenyl, 2H, d), 7.83-7.86 (phenyl, 1H, t), 7.75-7.79 (phenyl, 2H, t).

Next, 6.52 g of Compound 1 (22.6 mmol) and 30 ml of acetone were added into a reaction bottle. Next, potassium chloride (KCl) aqueous solution (6 g of potassium chloride dissolved in 30 ml of water) was added slowly into a reaction bottle under a nitrogen atmosphere. After stirring for 1 hr, potassium hydroxide (KOH) aqueous solution (4 g of potassium hydroxide dissolved in 10 ml of water) was added into the reaction bottle. Next, the reaction bottle was heated to reflux (100° C.), and the mixture was stirred for 20 hr. Next, after cooling, the result was extracted three times using 50 ml of water and 50 ml of hexane as the extraction solvent, and then the organic phase was collected. After drying, filtering and concentrating the organic phase, methyl phenyl sulfide with a yield of about 87% was obtained. The synthesis pathway of the above reaction was as follows:

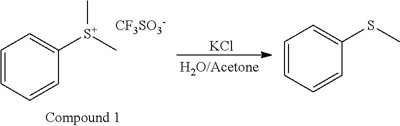

Methyl phenyl sulfide was analyzed by nuclear magnetic resonance (NMR) spectroscopy and the result is as follows: ¹H NMR (400 MHz, ppm, CDCl₃): 2.52 (—CH₃, 3H, s), 7.14-7.19 (phenyl, 1H, t), 7.31 (phenyl, 4H, m).

### EXAMPLE 2

2.4 g of diphenyl ether (14 mmol), 0.92 g of dimethyl sulfoxide (12 mmol), and 15 ml of dichloromethane were added into a reaction bottle, and then cooled to −35° C. under a nitrogen atmosphere. After stirring for several minutes, 2.3 ml of trifluoromethanesulfonic anhydride (14 mmol) was added into the reaction bottle. After stirring at 0° C. (ice bath) for 1 hr, a solution with white suspension was obtained. After stirring at room temperature for 12 hr, the result was concentrated and recrystallized with ethyl ether, obtaining Compound 2 with a yield of about 65%. The synthesis pathway of the above reaction was as follows:

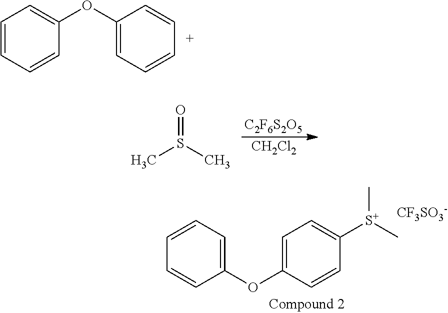

Compound 2 was analyzed by nuclear magnetic resonance (NMR) spectroscopy and the result is as follows: ¹H NMR (400 MHz, ppm, Acetone): 3.50 (—CH₃, 6H, s), 8.18 (phenyl, 2H, d), 7.52 (phenyl, 2H, t), 7.26-7.34 (phenyl, 3H, m), 7.18 (phenyl, 2H, d).

Next, 2.9 g of Compound 2 (7.6 mmol) and 10 ml of acetonitrile were added into a reaction bottle. Next, potassium chloride (KCl) aqueous solution (9 g of potassium chloride dissolved in 20 ml of water) was added slowly into the reaction bottle under a nitrogen atmosphere. After stirring for 1 hr, 0.22 g of KOH aqueous solution was added into the reaction bottle. Next, the reaction bottle was heated to reflux (100° C.), and the mixture was stirred for 17 hr. Next, after cooling, the result was extracted three times using 50 ml of water and 50 ml of hexane as the extraction solvent, and then the organic phase was collected. After drying, filtering and concentrating the organic phase, methyl(4-phenyloxy)phenyl sulfide with a yield of about 60% was obtained. The synthesis pathway of the above reaction was as follows:

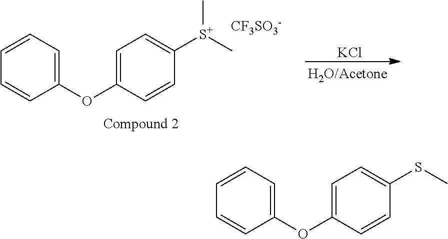

Methyl(4-phenyloxy)phenyl sulfide was analyzed by nuclear magnetic resonance (NMR) spectroscopy and the result is as follows: ¹H NMR (400 MHz, ppm, CDCl₃): 2.50 (—CH₃, 3H, s), 6.97-7.03 (phenyl, 4H, m), 7.15 (phenyl, 1H, t), 7.32-7.42 (phenyl, 4H, m).

It will be clear that various modifications and variations can be made to the disclosed methods and materials. It is intended that the specification and examples be considered as exemplary only, with the true scope of the disclosure being indicated by the following claims and their equivalents.

What is claimed is:

1. A method for preparing an aromatic sulfide or a salt thereof, comprising:
reacting a compound having a structure represented by Formula (I) to a compound having a structure represented by Formula (III) in the presence of a compound having a structure represented by Formula (II) to obtain a compound having a structure represented by Formula (IV) 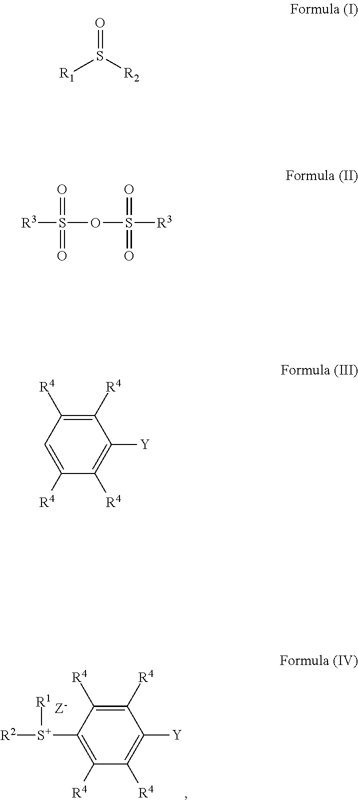
wherein R¹ and R² are independently C₁₋₆ alkyl group, or C₅₋₇ cycloalkyl group; R³ is independently C₁₋₆ alkyl group, C₅₋₇ cycloalkyl group, C₁₋₆ haloalkyl group, or aryl group; R⁴ is independently H, or C₁₋₆ alkyl group; Y is H, aryl group, or —X—R⁵; X is —O—, —NH—, —PH—, or —S—, or Y and an adjacent R⁴ are optionally combined with the carbon atoms to which they are attached attached, to form an aryl group; R⁵ is H, C₁₋₆ alkyl group, C₅₋₇ cycloalkyl group, or aryl group; and, Z⁻ is R³SO₃⁻.

2. The method as claimed in claim 1, wherein R¹ and R² are independently methyl, ethyl, propyl, isopropyl, n-butyl, t-butyl, sec-butyl, isobutyl, pentyl, hexyl, cyclopentyl, cyclohexyl, or cycloheptyl.

3. The method as claimed in claim 1, wherein R³ is independently methyl, ethyl, propyl, isopropyl, n-butyl, t-butyl, sec-butyl, isobutyl, pentyl, hexyl, fluoromethyl, fluoroethyl, fluoropropyl, cyclopentyl, cyclohexyl, or cycloheptyl.

4. The method as claimed in claim 1, wherein R⁴ is independently hydrogen, methyl, ethyl, propyl, isopropyl, n-butyl, t-butyl, sec-butyl, isobutyl, pentyl, or hexyl.

5. The method as claimed in claim 1, wherein the compound having a structure represented by Formula (I) is 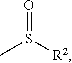
R² is C₁₋₆ alkyl group, or C₅₋₇ cycloalkyl group.

6. The method as claimed in claim 1, wherein the compound having a structure represented by Formula (I) is dimethyl sulfoxide, diethyl sulfoxide, methyl ethyl sulfoxide, methyl phenyl sulfoxide, or diphenyl sulfoxide.

7. The method as claimed in claim 1, wherein the compound having a structure represented by Formula (II) is methanesulfonic anhydride, ethanesulfonic anhydride, propanesulfonic anhydride, or trifluoromethanesulfonic anhydride.

8. The method as claimed in claim 1, wherein the compound having a structure represented by Formula (III) is 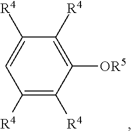
wherein R⁴ is independently hydrogen, or C₁₋₆ alkyl group; and, R⁵ is hydrogen, C₁₋₆ alkyl group, C₅₋₇ cycloalkyl group, or aryl group.

9. The method as claimed in claim 1, wherein the compound having a structure represented by Formula (III) is benzene, naphthalene, biphenyl, diphenyl amine, diphenyl sulfide, or diphenyl ether.

10. The method as claimed in claim 1, wherein the compound having a structure represented by Formula (IV) is 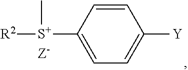
wherein R² is C₁₋₆ alkyl group, or C₅₋₇ cycloalkyl group; Y is hydrogen, aryl group, or —X—R⁵, wherein X is —O—, —NH—, —PH—, or —S—, or Y and an adjacent R⁴ are optionally combined with the carbon atoms to which they are attached, to form an aryl group; and R⁵ is H, C₁₋₆ alkyl group, C₅₋₇ cycloalkyl group, or aryl group; and Z⁻ is R³SO₃⁻.

11. The method as claimed in claim 1, further comprising:
reacting a nucleophile with the compound having a structure represented by Formula (IV), obtaining a compound having a structure represented by Formula (V), 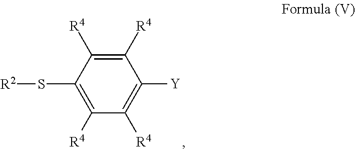
wherein R² is C₁₋₆ alkyl group, or C₅₋₇ cycloalkyl group; R⁴ is independently hydrogen, or C₁₋₆ alkyl group; Y is hydrogen, aryl group, or —X—R⁵, wherein X is —O—, —NH—, —PH—, or —S—, or Y and an adjacent R⁴ are optionally combined with the carbon atoms to which they are attached, to form an aryl group; and R⁵ is H, C₁₋₆ alkyl group, C₅₋₇ cycloalkyl group, or aryl group.

12. The method as claimed in claim 11, wherein the nucleophile is substituted or unsubstituted pyridine or derivatives thereof, amine, halogenated salt, alcohol, or amide.

13. The method as claimed in claim 11, wherein the nucleophile is pyridine, 4-methylpyridine, triethylamine, potassium chloride, methanol, ethanol, dimethylformamide, dimethylacetamide, N-methylpyrrolidone, or a combination thereof.

14. The method as claimed in claim 11, wherein the compound having a structure represented by Formula (V) is 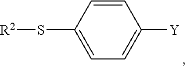
wherein R² is C₁₋₆ alkyl group, or C₅₋₇ cycloalkyl group; Y is hydrogen, aryl group, or —X—R⁵, wherein X is —O—, —NH—, —PH—, or —S—, or Y and an adjacent R⁴ are optionally combined with the carbon atoms to which they are attached, to form an aryl group; and R⁵ is H, C₁₋₆ alkyl group, C₅₋₇ cycloalkyl group, or aryl group.

15. The method as claimed in claim 11, wherein the compound having a structure represented by Formula (V) is [chemical structure]
